(19) United States
(12) Patent Application Publication
(10) Pub. No.: US 2016/0340377 A1
(43) Pub. Date: Nov. 24, 2016

(54) METHOD FOR PURIFYING OXIDIZED FORM OF BETA-NICOTINAMIDE ADENINE DINUCLEOTIDE

(71) Applicant: BONTAC BIO-ENGINEERING (SHENZHEN) CO., LTD, Shenzhen (CN)

(72) Inventors: Rongzhao FU, SHENZHEN (CN); Zhu DAI, SHENZHEN (CN); Qi ZHANG, SHENZHEN (CN)

(73) Assignee: BONTAC BIO-ENGINEERING (SHENZHEN) CO., LTD, SHENZHEN (CN)

(21) Appl. No.: 15/109,545

(22) PCT Filed: Dec. 4, 2015
(86) PCT No.: PCT/CN2015/096378

§ 371 Date: Jul. 1, 2016

(30) Foreign Application Priority Data
May 19, 2015 (CN) 201510255149.9

(51) Int. Cl. C07H 1/06; C07H 21/02

(52) U.S. Cl. CPC C07H 1/06; C07H 21/02

(57) ABSTRACT

The present invention discloses a method for purifying oxidized form of β-nicotinamide adenine dinucleotide, comprising the steps of: sequentially microfiltrating and nanofiltrating a reaction solution obtained after an enzymatic reaction by using filteration membranes, to collect a concentrate for use; then adding an acid to the concentrated filtrate, to adjust the pH of the concentrate, and purifying by gradient elution using a reverse-phase chromatographic column as a stationary phase, a buffer solution as a phase A, and ethanol as a phase B; and concentrating the purified solution by nanofiltrating it with a filtration membrance, and then freeze drying it in a vacuum freeze drier. In the present invention, the oxidized form of β-nicotinamide adenine dinucleotide is purified by reverse phase high performance liquid chromatography, such that the oxidized form of β-nicotinamide adenine dinucleotide has a high purity and high yield, thus meeting the requirements in industry.

### BACKGROUND

1. Technical Field

The present invention relates to a method for purifying a coenzyme, and particularly to a method for purifying oxidized form of β-nicotinamide adenine dinucleotide.

2. Related Art

Nicotinamide adenine dinucleotide (NAD) is also referred to as diphosphopyridine nucleotide (DPN), codehydrogenase I or coenzyme I. NAD receives a hydrogen atom and an electron from a substrate in the presence of various hydrogenases, and becomes a reduced form, in which pyridine is reduced. The process may take place reversibly. Therefore, NAD+ may serve as a substrate common to various hydrogenases. Upon action between two hydrogenases, the redox reaction (electron transfer) between two substrates can be catalyzed in the presence of a small amount of NAD+. NAD may be widely used as a raw material in chemical synthesis, and has a high market demand.

At present, the prevalent purification processes mainly include purification by means of ion exchange resins, recrystallization, and others. However, the production process is difficult to be controlled, the production efficiency is low, the product purity is only about 95%, and the yield is only 60%, thus being failed to meet the demand in the market.

Therefore, improvements and developments are needed in the art.

### SUMMARY

### Technical Problem

In view of the defects existing in the prior art, an object of the present invention is to provide a method for purifying oxidized form of β-nicotinamide adenine dinucleotide, for the purpose of addressing the problems of low purity, low yield, and limited production capability occurring to an existing process for purifying oxidized form of β-nicotinamide adenine dinucleotide.

### Technical Solution

To achieve the above object, the following technical solution is adopted in the present invention.

A method for purifying oxidized form of β-nicotinamide adenine dinucleotide comprises the steps of

a. sequentially microfiltrating and nanofiltrating a reaction solution obtained after an enzymatic reaction, to collect a concentrate for use;

b. then adding phosphoric acid or hydrochloric acid to the concentrate to adjust the pH to 3-5, and purifying by gradient elution using a reverse-phase chromatographic column as a stationary phase, a buffer solution as a phase A, and ethanol as a phase B; and

c. nanofiltrating the filtrate obtained in Step b, and finally freeze drying it in a vacuum freeze drier.

In the method for purifying oxidized form of β-nicotinamide adenine dinucleotide, the nanofiltration membrane used for nanofiltration in Step a is ahollow fiber membrane with a 200 cut-off molecular weight.

In the method for purifying oxidized form of β-nicotinamide adenine dinucleotide, the concentration of the concentrate in Step a is 30-50 g/L.

In the method for purifying oxidized form of β-nicotinamide adenine dinucleotide, the reverse-phase chromatographic column in Step b is octadecylsilane-bonded silica gel.

In the method for purifying oxidized form of β-nicotinamide adenine dinucleotide, the buffer solution in Step b is a 20 mM buffer solution formulated with formic acid and sodium hydroxide.

In the method for purifying oxidized form of β-nicotinamide adenine dinucleotide, the buffer solution in Step b has a pH of 3-5.

In the method for purifying oxidized form of β-nicotinamide adenine dinucleotide, the volume ratio of the phase A to the phase B in Step b is greater than 3:97, and less than 1.

In the method for purifying oxidized form of β-nicotinamide adenine dinucleotide, wherein the gradient elution time in Step b is 40 min.

In the method for purifying oxidized form of β-nicotinamide adenine dinucleotide, the detection wavelength in Step b is 260 nm.

In the method for purifying oxidized form of β-nicotinamide adenine dinucleotide, the concentration of the solution after concentration by nanofiltration in Step c is 100-150 g/L.

### Beneficial Effect

In the method for purifying oxidized form of β-nicotinamide adenine dinucleotide provided in the present invention, the oxidized form of nicotinamide adenine dinucleotide is purified by reverse phase high performance liquid chromatography. As a result, the purity of the resulting product is up to 99%, the yield is up to 90% or more, and the production efficiency is 1 time higher than that of other processes, thus greatly reducing the production cost, and meeting the requirements for production and price in the market. Accordingly, the process has a broad application prospect.

### DETAILED DESCRIPTION

The present invention provides a method for purifying oxidized form of β-nicotinamide adenine dinucleotide. To make the objects, technical solutions, and effects of the present invention clearer and more precise, the present invention is described in further detail hereinafter. It should be understood that the specific embodiments described herein are merely provided for illustrating, instead of limiting the present invention.

The present invention provides a method for purifying oxidized form of β-nicotinamide adenine dinucleotide, in which the oxidized form of β-nicotinamide adenine dinucleotide is purified by reverse phase high performance liquid chromatography, such that the pufified oxidized form of β-nicotinamide adenine dinucleotide has a high purity and a high yield, thus meeting the requirements in industry.

A method for purifying oxidized form of β-nicotinamide adenine dinucleotide comprises the steps of:

a. sequentially microfiltrating and nanofiltrating a reaction solution obtained after an enzymatic reaction, to collect a concentrate for use;

b. then adding phosphoric acid or hydrochloric acid to the concentrate to adjust the pH to 3-5, and purifying by gradient elution using a reverse-phase chromatographic column as a stationary phase, a buffer solution as a phase A, and ethanol as a phase B; and

c. nanofiltrating the filtrate obtained in Step b, and finally freeze drying it in a vacuum freeze drier.

In the present invention, the reaction solution obtained after an enzymatic reaction is firstly microfiltered in Step a, in which the microfiltration is carried out using a microfiltration membrance of 0.35 μm under an operation pressure of 0.1 Mpa, and the microfiltration is used to remove the microorganisms, because the microfiltration membrance allows macromolecules and dissolved inorganic salts to pass through, and retains microorganisms, bacteria, and suspended matter. Then, the filtrate obtained after microfiltration is nanofiltrated using a nanofiltration membrane, in which the nanofiltration membrane is a hollow fiber membrane, and preferably the nanofiltration membrane is a hollow fiber membrane with a 200 molecular weight cut-off. By using the nanofiltration membrane of this material, some dissolved salts and the organic compounds with a molecular weight of 200 or above can be removed, thereby further improving the purity and yield of the product.

In the present invention, the concentration of the concentrate in Step a is 30-50 g/L. In the present invention, the sample solution is treated by microfiltration and nanofiltration before injection, such that the particles, microorganisms, organic compounds and some dissolved inorganic salts are removed, to reduce the subsequent chromatographic elution time, and avoid the clogging of the column by particles, thereby extending the service life of the column. In the present invention, after concentration by microfiltration and nanofiltration in Step a, the concentration of the concentrate is 30-50 g/L. Concentrating the sample solution to such a concentration can facilitate the reduction of the sample elution time in Step b, and the improvement of the separation efficiency.

In the present invention, the reverse-phase chromatographic column in Step b is octadecylsilane-bonded silica gel. By using the nonpolar octadecylsilane-bonded silica gel as the stationary phase, the sample solution can be effectively and rapidly separated, and the resulting oxidized form of β-nicotinamide adenine dinucleotide has a high purity and a high yield.

Further, in Step b in the present invention, the octadecylsilane-bonded silica gel is further pretreated by reflux with HCl. By activating the octadecylsilane-bonded silica gel column with HCl, the Si—O—Si bond is broken, to form free Si—OH, thereby increasing the number of the Si—OH groups on the surface of the silica gel. This can facilitate the progressing of the bonding reaction, and the effect of chromatographic separation is more desirable.

In Step b in the present invention, the buffer solution is a 20 mM buffer solution formulated with formic acid and sodium hydroxide. The concentration of the buffer solution has a direct influence on the peak shape of a target component, thus affecting the separation effect of the chromatographic column. Where the concentration of the buffer solution is low, the chromatographic peak is caused to tail and broaden. Where the concentration of the buffer solution is high, the chromatographic column is damaged, and the service life of the chromatographic column is shortened. In the present invention, when the concentration of the buffer solution is 20 mM, the peak shape of the resulting chromatographic peak is better, and the effect of chromatographic separation is more preferable.

Further, in Step b in the present invention, the buffer solution has a pH of 3-5. The selection of a proper pH of the buffer solution is critical to a dissociable compound. An appropriate pH of the buffer solution may allows the dissociable compound to exist in one form, thereby facilitating the acquisition of a good and sharp peak, such that the separation effect is much better. In contrast, an improper pH may lead to the formation of a broad, asymmetric, and split peak. In the present invention, when the pH of the buffer solution is 3-5, a target peak with a good peak shape is obtained. Preferably, the pH of the buffer solution is 4, at which the peak shape of the target peak is optimum, and the separation effect is the most desirable.

Further, in Step b in the present invention, the volume ratio of the phase A to the phase B is greater than 3:97, and less than 1. Preferably, the volume ratio of the phase A to the phase B is greater than 20:80, and less than 40:60. In the range, the oxidized form of β-nicotinamide adenine dinucleotide can be well separated.

Further, in Step b in the present invention, the gradient B % is from 3 to 15%. In such a range, the purpose of rapidly separating the oxidized form of β-nicotinamide adenine dinucleotide with a good separation effect can be achieved with the mobile phases.

Further, in Step b in the present invention, the detection wavelength is 260 nm, because the oxidized form of β-nicotinamide adenine dinucleotide has a maximum absorption at this wavelength. Therefore, the chromatographic peak has a good peak shape, and the sensitivity is high.

In Step b in the present invention, the gradient elution time is 40 min. Because the ingredients in the concentrate are complex, if isocratic elution is employed, the elution time is long, the separation efficiency is poor, and the sensentivity is less good. In the present invention, the oxidized form of β-nicotinamide adenine dinucleotide is purified by gradient elution, such that the degree of separation is high, the separation time is short, the sensitivity is high, and the separation effect is good. The sample can be well separated when the gradient elution time is 40 min.

In Step c in the present invention, the product filtrate after salt change is concentrated to 100-150 g/L by nanofiltrating using a hollow fiber membrane with a 200 molecular weight cut-off, and then freeze dried in a vacuum freeze drier, to obtain a high-purity and high-yield freeze dried product.

In the present invention, the flow rate of the mobile phase is 50-3000 mL/min, and preferably 50-80 mL/min, 400-500 mL/min, or 2500-3000 mL/min. When the flow rate of the mobile phases is increased, the oxidized form of β-nicotinamide adenine dinucleotide can be rapidly separated with a good separation effect by the chromatographic column in the present invention.

In the present invention, the column diameter and length are 5 cm×30 cm, 15 cm×30 cm or 30 cm×30 cm.

The present invention is further described with reference to examples.

### Example 1

1. Sample treatment: A reaction solution obtained after an enzymatic reaction was sequentially microfiltrated and nanofiltrated. The microfiltration was carried out using a microfiltration membrance of 0.35 μm under an operation pressure of 0.1 Mpa, and the microfiltration was used to remove the microorganisms. The nanofiltration was carried out using a hollow fiber membrane with a 200 molecular weight cut-off, to concentrate the filtrate to 30-50 g/L. A concentrate was collected for use.

2. Purification:

Purification conditions: Chromatographic column: chromatographic column with octadecylsilane-bonded silica gel as a stationary phase, and column diameter and length: 5 cm×30 cm. Mobile phases: Phase A: 20 mM buffer solution pH 3 formulated with formic acid and sodium hydroxide; and Phase B: ethanol. Flow rate: 50-80 mL/min. Detection wavelength: 260 nm. Gradient: B %: 3%-15% (over an elution time of 40 min). Amount of injection: 10-15 g.

Purification process: Phosphoric acid or hydrochloric acid was added to the concentrated sample filtrate to adjust the pH to 3-5, and the chromatographic column was rinsed with 30 wt % or above of ethanol, equilibrated, and loaded with the sample in an amount of 10-15 g sample filtrate. The sample was eluted for 40 min with a linear gradient, and the target peak was collect.

3. Concentration and freeze drying: The purified filtrate was concentrated to 100-150 g/L by nanofiltrating using a nanofiltration membrane (hollow fiber membrane with a 200 molecular weight cut-off), and then freeze dried in a vacuum freeze drier, to obtain a freeze dried product with a purity that is higher than 99% and a total yield that can be up to 90.3%.

### Example 2

1. Sample treatment: A reaction solution obtained after an enzymatic reaction was sequentially microfiltrated and nanofiltrated. The microfiltration was carried out using a microfiltration membrance of 0.35 μm under an operation pressure of 0.1 Mpa, and the microfiltration was used to remove the microorganisms. The nanofiltration was carried out using a hollow fiber membrane with a 200 molecular weight cut-off, to concentrate the filtrate to 30-50 g/L. A concentrate was collected for use.

2. Purification:

Purification conditions: Chromatographic column: chromatographic column with octadecylsilane-bonded silica gel as a stationary phase, and column diameter and length: 15 cm×30 cm. Mobile phases: Phase A: 20 mM buffer solution pH 4 formulated with formic acid and sodium hydroxide; and Phase B: ethanol. Flow rate: 400-500 mL/min. Detection wavelength: 260 nm. Gradient: B %: 3%-15% (over an elution time of 40 min). Amount of injection: 80-100 g.

Purification process: Phosphoric acid or hydrochloric acid was added to the concentrate to adjust the pH to 3-5, and the chromatographic column was rinsed with 30 wt % or above of ethanol, equilibrated, and loaded with the sample in an amount of 80-100 g sample filtrate. The sample was eluted for 40 min with a linear gradient, and the target peak was collect.

3. Concentration and freeze drying: The purified filtrate was concentrated to 100-150 g/L by nanofiltrating using a nanofiltration membrane (hollow fiber membrane with a 200 molecular weight cut-off), and then freeze dried in a vacuum freeze drier, to obtain a freeze dried product with a purity that is higher than 99% and a total yield that can be up to 91.5%.

### Example 3

1. Sample treatment: A reaction solution obtained after an enzymatic reaction was sequentially microfiltrated and nanofiltrated. The microfiltration was carried out using a microfiltration membrance of 0.35 μm under an operation pressure of 0.1 Mpa, and the microfiltration was used to remove the microorganisms. The nanofiltration was carried out using a hollow fiber membrane with a 200 molecular weight cut-off, to concentrate the filtrate to 30-50 g/L. A concentrate was collected for use.

2. Purification:

Purification conditions: Chromatographic column: chromatographic column with octadecylsilane-bonded silica gel as a stationary phase, and column diameter and length: 30 cm×30 cm. Mobile phases: Phase A: 20 mM buffer solution pH 5 formulated with formic acid and sodium hydroxide; and Phase B: ethanol. Flow rate: 2500-3000 mL/min. Detection wavelength: 260 nm. Gradient: B %: 3%-15% (over an elution time of 40 min). Amount of injection: 400-500 g.

Purification process: 20 mM tetramethylammonium hydroxide was added to the concentrate to concentrate, and the chromatographic column was rinsed with 30 wt % or above of ethanol, equilibrated, and loaded with the sample in an amount of 400-500 g sample filtrate. The sample was eluted for 40 min with a linear gradient, and the target peak was collect.

3. Concentration and freeze drying: The purified solution was concentrated to 100-150 g/L by nanofiltrating using a nanofiltration membrane (hollow fiber membrane with a 200 molecular weight cut-off), and then freeze dried in a vacuum freeze drier, to obtain a freeze dried product with a purity that is higher than 99% and a total yield that can be up to 91.2%.

It can be known from the above examples that when the reverse phase high performance liquid chromatography is used to purify the oxidized form of β-nicotinamide adenine dinucleotide, the product obtained has a purity up to 99%, the yield is up to 90% or more, and the production efficiency is 1 time higher than that of other processes, thus greatly reducing the production cost, and meeting the requirements for production and price in the market. Accordingly, the process has a broad application prospect.

It should be understood that equivalent replacements or changes may be made by those ordinarily skilled in the art based on the the technical solution and concept of the present invention, which are all embraced in the protection scope as defined by the accompanying claims of the present invention.

What is claimed is:

1. A method for purifying oxidized form of β-nicotinamide adenine dinucleotide, comprising the steps of
a. sequentially microfiltrating and nanofiltrating a reaction solution obtained after an enzymatic reaction, to collect a concentrate for use;
b. then adding phosphoric acid or hydrochloric acid to the concentrate to adjust the pH to 3-5, and purifying by gradient elution using a reverse-phase chromatographic column as a stationary phase, a buffer solution as a phase A, and ethanol as a phase B; and
c. nanofiltrating the filtrate obtained in Step b, and finally freeze drying it in a vacuum freeze drier.

2. The method for purifying oxidized form of β-nicotinamide adenine dinucleotide according to claim 1, wherein the nanofiltration membrane used for nanofiltration in Step a is a hollow fiber membrane with a 200 molecular weight cut-off.

3. The method for purifying oxidized form of β-nicotinamide adenine dinucleotide according to claim 1, wherein the concentration of the concentrate in Step a is 30-50 g/L.

4. The method for purifying oxidized form of β-nicotinamide adenine dinucleotide according to claim 1, wherein the reverse-phase chromatographic column in Step b is octadecylsilane-bonded silica gel.

5. The method for purifying oxidized form of β-nicotinamide adenine dinucleotide according to claim 1, wherein the buffer solution in Step b is a 20 mM buffer solution formulated with formic acid and sodium hydroxide.

6. The method for purifying oxidized form of β-nicotinamide adenine dinucleotide according to claim 1, wherein the buffer solution in Step b has a pH of 3-5.

7. The method for purifying oxidized form of β-nicotinamide adenine dinucleotide according to claim 1, wherein the volume ratio of the phase A to the phase B in Step b is greater than 3:97, and less than 1.

8. The method for purifying oxidized form of β-nicotinamide adenine dinucleotide according to claim 1, wherein the gradient elution time in Step b is 40 min.

9. The method for purifying oxidized form of β-nicotinamide adenine dinucleotide according to claim 1, wherein the detection wavelength in Step b is 260 nm.

10. The method for purifying oxidized form of β-nicotinamide adenine dinucleotide according to claim 1, wherein the concentration of the solution after concentration by nanofiltration in Step c is 100-150 g/L.
